SEQUENCE LISTING (1 sequence)

SEQ ID NO: 1 - protein, 494 aa, Salmonella typhimurium
  conserved region (1)..(190): Flagellin FliC
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

(19) United States
(12) Patent Application Publication
(10) Pub. No.: US 2006/0257415 A1
(43) Pub. Date: Nov. 16, 2006

(54) Adjuvants

(75) Inventors: Jean-Claude Sirard, Villeneuve d'Ascq (FR); Jean-Pierre Kraehenbuhl, Rivaz (CH)

Correspondence Address: NIXON & VANDERHYE, PC, 901 NORTH GLEBE ROAD, 11TH FLOOR, ARLINGTON, VA, 22203, US

(73) Assignee: FONDATION EUROV ACC, Lausanne (CH)

(21) Appl. No.: 10/526,271

(22) PCT Filed: Sep. 3, 2003
(86) PCT No.: PCT/GB03/03797

§ 371 Date: Jun. 2, 2005

Related U.S. Application Data
(60) Provisional application No. 60/407,294, filed on Sep. 3, 2002.

(51) Int. Cl. A61K 39/00; A61K 39/38

(52) U.S. Cl. 424/184.1

(57) ABSTRACT

A method is provided for inducing the adaptive immune response of a patient to an antigen comprising administering to the patient an effective amount of a flagellin protein or peptide fragment thereof to induce an adaptive immune response to said antigen.

The present invention relates to stimulation or induction of a patient's immune response to an antigen, particularly an adaptive immune response in mucosal tissues, such as intestinal epithelial cells. The invention also provides novel mutated and truncated flagellin proteins and nucleic acids encoding for these.

The gut represents a unique niche for bacteria of the normal flora and correspondingly for enteropathogenic microbes. The intestinal epithelium constitutes not only the physical barrier that separates the lumenal environment from the host milieu, but it also acts as a sentinel sensing injuries in the intestinal tract. Enteropathogenic bacteria colonize the epithelium and their intimate interaction with the epithelial cells activates proinflammatory signaling pathways (1-3). This innate response is essential for rapid clearance of bacteria Adaptive immunity is also stimulated to prevent re-infection, but the mechanisms initiating this response in the gut epithelium have yet not been identified.

Dendritic cells (DC) are bone marrow-derived antigen-presenting cells with the unique ability to induce primary immune responses. The recruitment of DCs into the epithelium is therefore a prerequisite to initiate an adaptive response. The trafficking of DCs depends on differential expression of CCR6 and CCR7 chemokine receptors (4-7). The CCL20 chemokine also known as LARC, MIP-3 alpha and Exodus is the ligand of CCR6 receptor (8). Immature DCs express CCR6 and efficiently take up soluble and particulate antigens (for review, 9). Maturation of DCs is induced by danger signals, i.e. bacterial, viral, or cellular components, and is characterized by the upregulation of antigen presentation, co-stimulatory molecules, and of the CCR7 chemokine receptor that mediates migration of activated DCs to the draining lymph nodes. The CCL20 gene is expressed in the epithelium over murine Peyer's patches and colon and in human colon, appendix, tonsils, and skin keratinocytes (4, 6-8, 10, 11). CCR6-expressing DCs are found in tissues close to CCL20-expressing epithelial cells or keratinocytes (6, 7, 10). In CCR6-knockout mice, subepithelial myeloid DCs are absent in the dome of Peyer's patches and mucosal immune responses are impaired (7). These findings have emphasized the instrumental role of CCL20-dependent DC trafficking in induction of adaptive responses in the gut.

Enteropathogens compete with the normal flora and produce specific virulence factors to overcome innate defences. Enteroinvasive bacteria (e.g., Salmonella, Shigella, Yersinia, Listeria) adhere and invade the epithelium via M cells of Peyer's patches (12). After subepithelial translocation, they invade enterocytes or phagocytes and/or replicate. Invasive bacteria divert cellular signalling by interacting with cell surface receptors or with cytosolic targets using toxins injected into the cell cytoplasm via a type III secretion system (3, 13). Salmonella enterica of various serotypes provoke gastroenteritis in mammals characterized by mucosal inflammation and diarrhea. Salmonella are the only bacteria that can invade apically enterocytes along the crypt to villus axis of the small intestine (14). In human intestinal epithelial cells, the Salmonella-induced inflammatory response is characterized by basal secretion of IL-8 (CXCL8) and of various pro-inflammatory chemokines that recruit neutrophils in the subepithelial compartment (1, 15, 16). The induction of IL-8 secretion depends on virulence factors of Salmonella and on epithelial NF-κB signaling (1, 2, 17, 18).

The present inventors have investigated whether the release of intestinal epithelial chemokines in response to bacteria is able to recruit immune cells that initiate adaptive immunity. They have previously reported, see PNAS 98 (24) 13722-13727, Nov. 20^{th} 2001, that S. typhimurium flagellins stimulate the secretion of the CCL20 chemokine from epithelial cells, which triggers DC chemotaxis. They also observed that expression of the pro-inflammatory chemokine IL-8 is induced by flagellin in the intestinal epithelial cells, (see also Gewirtz et al).

The present inventors have determined that flagellin, and particularly Salmonella flagellin, induce the direct maturation of dendritic cells, as shown by upregulation of costimulatory molecules and antigen presenting functions for MHC class II-restricted responses. Dendritic cells undergo maturation as a prerequisite for optimal and effective presention of antigen to lymphocytes.

The present inventors have determined thus that flagellin, and particularly Salmonella flagellin, may be used to induce the immune response. They have demonstrated this in peripheral and mucosal tissues, after subcutaneous and intranasal routes of immunization. The present invention provides use of flagellin and homologues thereof (mutated or truncated or peptides that are fragments thereof) to stimulate signalling in epithelial cells, and directly on dendritic cells, resulting in increased antibody and cell-mediated immune responses in systemic and mucosal compartment.

The present invention further provides modified, ie mutated or truncated flagellins. Such modified flagellins would bind to the activation sites, ie. Toll-Like Receptors (TLRs) or TLR-associated co-receptors on dendritic cells and epithelial cells to activate them, acting as signalling molecules for these receptors.

The inventors have determined that Salmonella flagellins specifically stimulate CCL20 chemokine expression and secretion by epithelial intestinal cells resulting in chemotaxis of immature dendritic cells. Such DC migration could be essential for uptake of flagellated enteropathogens followed by antigen processing and presentation necessary for the induction of an adaptive immune response in the gut.

Furthermore, flagellin, when injected subcutaneously with MHC class I epitope, stimulates CD8+ lymphocytes to produce IFN-γ, suggesting that upregulation of costimulatory molecules on dendritic cells is sufficient to activate Cytotoxic T Lymphocyte (CTL) functions directed against peptides loaded on MHC class I molecules on dendritic cells. Flagellin is, therefore, particularly useful as an adjuvant for CD8 immune responses against coadministered MHC class I-restricted peptides.

Flagellin is widely distributed and conserved among distant bacterial species (25). The domain involved in cell signalling is shared by S. typhimuriuim FliC and FljB and S. enteritidis FliC molecules indicating that it is located in conserved regions, i.e. 170 amino- and 90 carboxy-terminal residues.

Using genetic and biochemical strategies, the inventors have now determined that the amino and carboxy terminal regions are required for cell signalling. The central region (between residues 191 and 353), which is variable among flagellins from various Salmonella serotypes and from various bacterial species, does not play a role in cell signalling.

Flagellins from various Gram negative or positive bacteria, including L. monocytogenes, are pro-inflammatory in the picomolar range (inventor's observations and (21, 24, 26-28)). Therefore, flagellin presents all features of pathogen-associated molecular patterns (PAMP).

Toll-like receptors (TLR) are involved in signal transduction of mammalian, plant and insect PAMPs (29). Recently, TLR5 has been shown to mediate flagellin-dependent signalling in transfected mammalian cells (28). TLR5 is expressed in Caco-2 cells (30), suggesting that in the gut, flagellin could trigger chemokine expression via TLR5. Moreover, in human intestine, TLR5 is detected on the apical and basal surfaces of enterocytes (30). LPS, which signals injury in peripheral tissues or in sterile mucosal tissues, is inactive in the gut lumen where the Gram negative bacteria are abundant. The gut has developed detection system for danger using other PAMPs. Flagellin is one PAMP candidate, but other bacterial factors are involved in mucosal cell signalling, for instance E. coli P fimbriae in urinary epithelia triggers inflammation via TLR4 (31).

The inventors have shown that various enteropathogenic but not commensal bacteria stimulated CCL20 and IL-8 gene expression. In pathogens, flagella are expressed during infection and the associated-motility is crucial for virulence (3). Pathogenic bacteria produce also virulence factors for specific adhesion, and/or invasion, and/or injury of epithelial cells (3). Commensal bacteria can also be equipped with flagella. However, even if expressed in vivo, the flagella of commensal bacteria are probably not contacting the epithelial cells. The microbial flora is confined to luminal compartment and mucus layer (32). We propose that in vivo, only enteropathogenic bacteria could bring flagellin in close contact to the epithelial cell surface resulting in induction of cell signalling. Alternatively, non-pathogenic bacteria have been shown to downregulate the pro-inflammatory cascade in epithelium (33), a mechanism that could also result in absence of flagellin-mediated signalling.

The gut is tolerant to most lumenal material including resident bacteria. Under steady state conditions, immature DCs are continually entering the gut probably via a constitutive CCL20-dependent mechanism and are sampling antigens (4, 7). The absence of injury and/or the anti-inflammatory environment of the gut have been proposed to induce tolerance (for review, 34) since antigen presentation by DCs occurs in absence of co-stimulation. The coupling of CCL20 and IL-8 transcriptional activation could be crucial for induction of protective immune responses in the gut. Flagellin was already known to induce the pro-inflammatory IL-8 chemokine expression in epithelial cells (21, 24, 27). The resulting inflammation provides danger signals, especially TNF-alpha and IL-1 cytokines, required for DC maturation. Thus, DCs attracted upon flagellin-stimulation may be fully activated and potent stimulators for adaptive responses. The recruitment of memory CD4 and B lymphocytes by CCL20 could also contribute to immunity in the gut (35).

Transcriptional activation of IL-8 and CCL20 genes is mediated by NF-κB (p65/p65 and p50/p65) (11, 18). A p65 binding site is present in the regulatory sequences of both IL-8 (18) and CCL20 genes (contig NT022115.2, -150 bp from ATG). This is consistent with the flagellin-dependent TLR5-mediated NF-κB signaling (28). The coupling of IL-8 and CCL20 expression is however not absolute. IL-8 gene transcription is significantly higher in epithelial cells exposed to live Salmonella compared to heat-killed bacteria or to flagellin whereas CCL20 mRNA levels remain the same. Therefore, activation of CCL20 expression seems to depend uniquely on flagellin, while IL-8 transcription is modulated by other components delivered by live bacteria as described previously (1, 2, 33).

Immature DCs recruited upon interaction of enteropathogenic microbes with epithelial cells could constitute an appropriate niche for bacterial survival and dissemination. S. typhimurium are taken up in Peyer's patches by subepithelial DCs (36). The survival of S. typhimurium in DCs is independent on virulence factors required for intracellular survival in macrophages (37). Therefore, the subepithelial immature DCs are the most potent candidate to carry the bacteria from the intestine to deeper organs such as mesenteric lymph nodes, spleen or liver, where it is transferred to macrophages. The chemokine-stimulating activity of flagellin could be essential to enhance migration of DCs into subepithelial areas of Peyer's patches and villi. Dissemination via DCs has been documented for L. monocytogenes (38).

Like Salmonella, L. monocytogenes produces flagella that are coordinately expressed with other virulence factors. Whether Listeria flagella are induction factors for CCL20 and whether immature DCs are vehicles for these bacteria are important questions to address for pathogenicity. Recently, Rescigno and coworkers reported that, both in vitro and in vivo, mouse DCs penetrate intestinal epithelium to sample lumenal bacteria (39). It remains to be tested whether this process is flagellin- and CCL20-mediated since the rapid migration of DCs do not parallel the CCL20 induction observed in Caco-2 cells. However, in mouse, administration of ileal ligated loops with flagellin results in induction of CCL20 gene transcription in epithelium of Peyer's patches and villi (FIG. 10). These observations show that flagellin effect in the gut epithelium is physiologically relevant.

The ability of flagellin signalling in innate and adaptive immunity provides new prospects in mucosal vaccination.

Monomeric flagellin signals in epithelial intestinal cells which results in CCL20-mediated recruitment of dendritic cells. Recruitment of dendritic cells at mucosal sites allows efficient uptake, processing, and presentation of antigens and vaccines in the draining lymph node. Therefore flagellin functions such as to enhance immune responses to co-administered antigens.

CCL20 seems to be instrumental for mucosal immune responses since CCR6 (receptor for CCL20 found on immature dendritic cell) knockout mice are impaired in such responses. Moreover, the trafficking of DC is known to be essential for mounting adaptive immune responses.

Thus the inventors have now determined that flagellin or fragments of flagellin can be used to induce the adaptive immune response via epithelial cells and dendritic cells, e.g. stimulate, increase or initiate an adaptive immune response to an antigen. The antigen may be any target antigen to which it is desired to induce or enhance an immune response, the target antigen may be present in the body of the patient, e.g. pathogenic microorganisms, or it may be administered to the patient, e.g. in the form of a vaccine.

Thus the present invention provides a method of inducing an adaptive immune response in a patient to a target antigen comprising administering to the patient a flagellin protein, or a peptide fragment thereof, in an amount effective to induce said response.

Preferably the first aspect also provides a method of inducing the adaptive immune response of a patient to a target antigen comprising administering to the patient an effective amount of flagellin protein or peptide fragment thereof capable of directly inducing the dendritic cell-dependent adaptive immune response or indirectly by recruiting dendritic cells at mucosal surfaces via the stimulation of epithelial cells. More preferably the first aspect provides a method of inducing the cell adaptive immune response of a patient to a target antigen comprising administering to the patient an effective amount of a flagellin protein or peptide fragment thereof capable of directly inducing the dendritic cell-dependent adaptive immune response wherein dendritic cell maturation is induced, more preferably increased.

A second preferred aspect of the present invention provides a method for inducing the adaptive immune response in the gut mucosa or more generally in any mucosal epithelium of a patient to a target antigen comprising administering to the patient an effective amount of a flagellin protein or peptide fragment thereof having an agonistic effect on CCL20 release.

Particularly the first and second preferred aspects of the present invention provide a method of inducing the adaptive immune response to a target antigen wherein the induction is by recruitment of immature dendritic cells. Preferably the flagellin or peptide fragment thereof used in the first and second preferred aspects is administered parenterally or transdermally. More preferably the flagellin or peptide fragment thereof used in the first and second preferred aspects is administered via the mucosal route; oral delivery is particularly preferred; still more preferably intranasal delivery. The flagellin or peptide fragment thereof may, for example, be administered alone, or in series, or co-administered, with a target antigen, particularly in the form of a vaccine adjuvant.

In a first subset of the first and second preferred aspect of the present invention there is provided a method for inducing recruitment of immature dendritic cells in oral vaccination, or intranasal vaccination, such as to induce an adaptive immune response comprising administering a flagellin protein or peptide fragment thereof with an antigen to which it is desired to induce said response.

Preferably the flagellin protein used in the first and second aspects of the invention includes at least one of the conserved regions of the N terminal sequence or the C terminal sequence of flagellin. More preferably the flagellin protein used in the first and second aspects of the invention includes at least one of the conserved regions of the 170 N terminal sequence and the 90 C terminal sequence of Salmonella flagellin, particularly the conserved regions shared by S. enteritidis or S. typhimurium. Preliminary results suggest that the region having this activity can be restricted to residues 1-190 and 354-494 of S typhimurium.

Particularly is provided use of such flagellin protein as a vaccine adjuvant.

Preferred peptide sequences for agents for use in the first subset are from 10 to 60 amino acids long, more preferably of 20 to 45 amino acids long and have high homology, e.g. 70% or more, more preferably 90% or more, to the corresponding parts of the sequences described herein as being involved in signalling in Caco-2 cells (See FIG. 8). More preferably the sequences have 70% or more identity to said parts of sequences.

Descriptions of homology and identity and how this may be determined will be well known to those skilled in the art. Particular interpretations may be as described in PCT/EP00/09325, and its corresponding US filing derived therefrom which is incorporated herein by reference. Homology and identity can be determined also by matching amino acids in order between sequences with introduction of gaps or deletions as required.

In a third aspect of the present invention there is provided use of a flagellin protein or peptide fragment thereof in the manufacture of a medicament for inducing recruitment of immature dendritic cells in mucosal vaccination such as to induce an adaptive immune response; more preferably oral or intranasal vaccination. Thus a preferred use is as an adjuvant, e.g. in an oral or intranasal vaccine. In a subset of the third aspect there is provided use of a flagellin or a peptide fragment thereof for inducing an adaptive immune response to an MHC Class I restricted peptide in subcutaneous vaccination.

A fourth aspect of the present invention provides a flagellin protein or peptide fragment thereof having agonistic effect on one or both of (a) CCL20 release from gut epithelial cells and (b) dendritic cell maturation, for use in therapy characterised in that the protein or peptide is truncated, mutated or has deletions therein which allow it to retain its ability to induce the immune response.

One preferred protein or peptide fragment of the fourth aspect retains the ability to bind to intestinal or dendritic flagellin receptors, e.g. TLR and associated receptors, and retain immune signalling.

Preferred proteins and peptides for the second, third and still further aspects will be as set out above for the first and second aspects.

A fifth aspect of the present invention provides a composition comprising a protein or peptide fragment of the invention together with a pharmaceutically acceptable carrier, excipient or diluent, or such protein or fragment in sterile and pyrogen free form.

A sixth aspect of the present invention provides a method for producing an inducer of the intestinal epithelial immune response comprising producing a protein or peptide fragment thereof that corresponds to the S. typhimurium flagellin amino acid sequence but which has been mutated, deleted or truncated such as to retain intestinal flagellin receptor binding properties while having active immune signalling properties.

Preferred methods of the sixth aspect comprise producing a DNA encoding for said mutated, deleted or truncated flagellin, e.g. by use of site directed mutation PCR primers.

The present invention will now be described by way of illustration only by reference to the following non-limiting examples. Further embodiments falling within the scope of the invention will occur to those skilled in the art in the light of these.

### FIGURES

FIG. 1. S. typhimurium-regulated expression of CCL20 gene in epithelial cells. Caco-2 cells in Transwell cultures were infected apically for 45 min with S. typhimurium ATCC14028 (moi=100), washed and incubated for the indicated times in gentamicin-supplemented medium. (a) Transcriptional activation of CCL20 gene: Total RNA was extracted and reverse transcribed. CCL20 mRNA levels were quantified using real-time PCR and 18S rRNA amplicons as standards. Values were expressed as relative increase of CCL20 mRNA quantity compared to non infected Caco-2 cells. (b) Secretion of CCL20 chemokine in basal culture medium. CCL20 concentration was measured by CCL20-specific ELISA on cell culture medium of Caco-2 cells.

FIG. 2. Pathogen-specific induction of CCL20 transcription in epithelial cells. Monolayers of Caco-2 cells were exposed apically for 45 min to bacterial strains (moi=100) and incubated for 2.5 h in gentamicin-containing medium. CCL20 expression was quantified by real-time RT-PCR. ATCC14028 was used as a positive control of CCL20 induction. Results are representative of at least 2 independent experiments. CCL20 transcription was analyzed upon exposure to (a) laboratory E. coli DH5α, (b) bacteria from human colon flora: E. coli EMO, B. vulgattus, and B. bifidum, and (c) enteroinvasive bacteria: S. eniteritidis SE857 and L. monocytogenes LO28.

FIG. 3. Salmonella induction factor for CCL20 expression is a heat stable secreted protein. Polarized Caco-2 cells were exposed apically for 45 min to bacteria (moi=100) (a). Then, cells were incubated for 2.5 h in gentamicin-supplemented medium. Alternatively, cells were exposed for 3.25 h to bacterial products at the indicated concentrations (b, c). Activation of CCL20 gene transcription was quantified by real-time RT-PCR. Results are representative of at least 3 independent experiments. (a) Induction of CCL20 transcription is independent on Salmonella-mediated invasion. (b) LPS-independent CCL20 transcription. Epithelial cells were treated apically or basally with 10 μg/ml of LPS from S. typhimurium. (c) Induction factor is a Salmonella secreted protein. Cells were exposed apically to 100 μl supernatants from S. typhimurium, heat-treated supernatant, or trypsin-digested and heat-treated supernatant. LB broth treated in the same conditions was used as control.

FIG. 4. Salmonella flagellins are inducing factors of CCL20 and IL-8 transcription in epithelial cells. Polarized Caco-2 cells were treated apically with bacteria (moi=100) or flagellin. CCL20 and IL-8 gene transcription was quantified by real-time RT-PCR (a, b). Results are representative of at least 3 independent experiments. (a) Cells were infected for 45 min with S. enteritidis, the fliC mutant SEFK32, or SEFK32(pRP2) (complemented with the FliC flagellin of S. typhimurium) and incubated for 2.5 h in gentamicin-containing medium. (b) Dose-dependent induction of CCL20 and IL-8 expression by flagellin. Cells were exposed apically for 3.25 h to purified S. typhimurium FliC flagellin at the indicated concentrations. (c) Flagellin expression in Salmonella strains. Supernatants (0.5 ml) from Salmonella cultures or purified S. typhimurium FliC flagellin (1 μg) were analyzed after SDS-PAGE by Coomassie blue staining (upper panel) and by immunoblotting (lower panel) with flagellin-specific Ab. Arrow and asterisk indicate the position of flagellins from ATCC14028 (52 KDa) and SE857 (56 KDa), respectively. Agglutination with flagellin-specific antibody was performed on bacteria grown in the same conditions.

FIG. 5. Immature DCs migrate in response to medium from flagellin-treated epithelial cells. rhCCL20 (7 ng/ml), control medium or basal medium of untreated or of flagellin-treated Caco-2 cells (7 ng/ml of CCL20) were used in migration assays of immature DCs. When specified, CCL20-specific mAb was mixed with medium 30 min before assay to neutralize CCL20. Results are representative of 2 independent experiments.

FIG. 6. TLR-5 expression-see legend on figure.

FIG. 7. Induction of CCL20 transcription in epithelial cells by Salmonella flagellin.

FIG. 8. The amino acid sequence of flagellin FliC from Salmonella typhimurium. Bold underlined residues indicate the proposed signalling region. EMBL accession number for the whole flagellin sequence is D13689, from which the encoding DNA is also available.

FIG. 9. Histogram of TLR5 mRNA levels of various cell types isolated from mouse spleen as determined by RT-PCR.

FIG. 10. Flagellin induces CCL20 transcription in vivo in mouse small intestine villi and Peyer's patches. BALB/c mice were anesthetized and ligated loops of small intestine (ileum) were prepared. Loops were injected intralumenally with 100 μg flagellin or 100 μg ovalbumin as negative control (in 200 μl PBS). Two hours later, mice sacrificed and ileal loops were frozen to prepare thin sections oif tissues. Hybridization with radiolabeled antisense CCL20 RNA was performed. After development, section were counterstained and observed under light microscope. Region containing a Peyer,s patch or villi are shown.

FIG. 11. IFN-γ release by lymphoid cells in response to flagellin, oil and control adjuvants with T helper peptide.

FIGS. 12A and 12B Flow cytometry results showing that dendritic cells are specifically activated by flagellin.

FIG. 13 Flagellin induces maturation of dendritic cells in-vitro. Flagellin is active at a low concentration, 10 ng/ml.

FIG. 14 Flow cytometry results showing maturation of dendritic cells in-vivo in response to flagellin.

FIG. 15 Histogram of anti-ovalbumin and anti-flagellin antibody titre in serum from mice immunized with flagellin, OVA, LPS or trypsin digested flagellin.

FIGS. 16A and 16B Histograms of serum antibody response to flagellin and OVA in BALB C mice (FIG. 16A) and NMRI mice (FIG. 16B).

FIG. 17 Histogram of IFN-γ release by spleen cells in response to ovalbumin, ovalbumin and flagellin, and ovalbumin and T helper petide in IFA.

FIG. 18 Graph showing ovalbumin specific antibody titre in serum sample from mice immunized with flagellin via the mucosal route (intranasal). Mice were immunized on days 0 and 21 via the intranasal route with ovalbumin or ovalbumin and flagellin. Ovalbumin specific antibody response was measured on Day 28 serum samples by ELISA.

FIG. 19 Graph showing ability of flagellin to signal (via induction of CCL20) in Caco 2 cells stably transfected with CCL20 luciferase reporter construct. All trypsin fragments deleted from the conserved distal regions 1-52 and 451-494 are devoid of signaling activity.

FIG. 20 Graph showing quantitative analysis of cell signalling of flagellin trypsin fragments in Caco 2 cells stably transfected with CCL20 luciferase reporter construct. Trypsin fragments deleted from any of the conserved distal regions 1-58, 462494, and 487-494 are strongly impaired in signaling activity.

FIG. 21 Graph showing impaired signalling of flagellin mutants (genetically engineered truncated forms) in Caco 2 cells stably transfected with CCL20 luciferase reporter construct. Among truncated mutants genererated by genetic engineering, only the molecule delta 191-353 is signaling.

FIG. 22 Graph showing quantitiative analysis of cell signalling of flagellin point mutants and deleted forms. All flagellin point mutants and deleted forms in the conserved region are signaling in the same concentration range as wild type molecule.

### EXAMPLES

### Methods

Bacterial strains and culture conditions. The bacterial strains are listed in Table 1. SIN strains were obtained by phage P22 HT105/int-1 transduction. Salmonella or E. coli were grown in Luria-Bertani (LB) broth for 24 h at 37° C., then diluted 1/1'000 in LB broth and grown in standing conditions for 18 h at 37° C. (19). Bacterial concentration was estimated to 10⁹ bacteria per ml per OD unit at 600 nm and calculated by plating. Ampicillin and kanamycin were added at 100 and 40 μg/ml, respectively. L. monocytogenes was grown in brain heart infusion medium (BHI) at 37° C., B. bifidum and B. vulgatus in BHI at 37° C. in anaerobic GasPak™ (Becton Dickinson, Cockeysville) jar using a glycerol frozen inoculum. Supernatants were filtered to remove residual bacteria and proteolysis was performed at 37° C. for 30 min with trypsin 10 μg/ml (Worthington Biochemical Corporation, Lakewood). When specified, bacteria or supernatants were heat-treated for 20 min at 65° C. For complementation, the ampicillin resistant plasmid pRP2 harboring an EcoRI fragment with S. typhimurium fliC genes (gift of K. Hughes) was introduced in Salmonella. Flagellin expression was checked by (i) agglutination with rabbit Salmonella H antiserum poly a-z (Difco laboratories, Detroit), (ii) motility in 0.35% agar, and (iii) by SDS-PAGE analysis of supernatants and immunoblot with poly a-z serum and peroxidase-conjugated anti-rabbit serum (Sigma, St. Louis).

Cell culture and stimulation. The human colon adenocarcinoma cell line Caco-2 clone 1 was grown in DMEM with glutamax, 10% FCS, 1% non-essential amino acids and 4 μg/ml transferrin (cell culture products from Gibco BRL, Rockville). T-84 intestinal epithelial cell line was grown in 50% DMEM, 50% Ham's F12 medium, 10% FCS and 2 mM L-glutamine. Cells were grown for 10 days at 37° C. under 5% CO₂ on Transwells (6 mm diameter, 3 μm pore, Corning Inc., Acton). The average of transepithelial electrical resistance was 450 Ω cm² and 1000 Ω cm² for Caco-2 and T-84 cells, respectively. Differentiation was also checked by the presence of apical microvilli and by upregulation of apical sucrase isomaltase with specific antibodies (gift from A. Zweibaum) using electron and confocal microscopy. Bacteria or bioactive materials were suspended in complete DMEM and added either apically (300 μl) or basally (1 ml). For infection, cells were incubated for 45 min with 10⁸ bacteria, i.e. a multiplicity of infection (moi) of ˜100, washed with PBS, and incubated with medium containing 50 μg/ml gentamicin (5 μg/ml for Listeria) to kill extracellular bacteria. Alternatively, cells were exposed to supernatant, lipopolysaccharide (LPS) or flagellin for the duration of experiment. At indicated times, total RNA was prepared and/or culture medium were recovered.

Real-time quantitative PCR for analysis of mRNA levels. Total RNA was isolated from cells of 3 Transwell filters (Rneasy, Quiagen, Switzerland) and reverse transcription (RT) was performed on 100 ng using Superscript II (Gibco BRL). Resulting cDNA (1 ng) was amplified in triplicates by the SYBR®-Green PCR assay, and products were detected on a Prism 5700 detection system (SDS, ABI/Perkin-Elmer, Fooster City). PCR reactions were incubated for 2 min at 50° C. and for 10 min at 95° C., followed by 40 amplification cycles with 1 min annealing/extension at 60° C. and 15s denaturation at 95° C. The 18S ribosomal RNA was used to standardize the total amount of cDNA. The primers for CCL20 (CCAAGAGTTTGCTCCTGGCT and TGCTTGCTGCTTCTGATTCG), IL-8 (CACCGGAAGGAACCATCTCA and GGAAGGCTGCCAAGAGAGC), and 18S (ACATCCAAGG AAGGCAGCAG and TTTTCGTCACTACCTCCCCG) designed from sequences (NM004591, Y00787, and X03205) yielded PCR products of 75, 72 and 65 bp, respectively. Specificity of PCR was checked by analyzing melting curves and sequencing. Relative mRNA levels (2^{ΔΔC}) were determined by comparing (i) the PCR cycle threshold (C) between cDNA of the gene of interest and of 18S rRNA (ΔC), (ii) ΔC values between treated and untreated conditions (ΔΔC). SD of relative mRNA levels were calculated as follows: 2(ΔΔC±√{SD[ΔC_{treated]}²+SD[ΔC_{untreated}]²}). Increase of RNA levels lower than 2 fold were not considered as significant.

CCL20-specific ELISA. Microplates coated with 3 μg/ml human CCL20-specific mAb (clone 67310.111, R&D Systems, Minneapolis) were used to capture CCL20 in culture medium. Goat anti-human CCL20 (R&D Systems) diluted at 1 μg/ml was used as the detection Ab and development was performed with peroxidase-conjugated rabbit anti-goat Ab (Sigma) diluted 1/2'000. CCL20 concentration was calculated from a standard curve using recombinant human (rh) CCL20 (R&D Systems). The detection threshold was 0.5 ng/ml.

LPS and flagellin purification. LPS was purified by hot phenol extraction as described previously (20). Alternatively, commercial S. typhimurium LPS was used (L-6511, Sigma). Flagellin was prepared from Salmonella strain SEFK32(pRP2) grown for 16 h at 37° C. with agitation in LB as described previously (21). Briefly, flagella were sheared from surface, pelleted by ultracentrifugation, and acidified to release flagellin monomers. Flagellin was concentrated in PBS and stored at −80° C.

Generation of CD34⁺-derived DCs. Progenitors were isolated from umbilical cord blood by positive selection using anti-CD34 mAb (Immu-133.3, Immunotech, France), goat anti-mouse IgG-coated microbeads and MidiMacs columns (Miltenyi Biotec, Germany). CD34⁺ cells were grown in RPMI-1640, 10% FCS, 200 U/ml rhGM-CSF (Schering-Plough Research Institute, Kenilworth), 50 U/ml rhTHFα (PeproTech Inc., Rocky Hill) and 10 U/ml rhSCF (R&D Systems). After 7 days, the cells (30-50% CD1a⁺ DCs, 25-35% CD1a⁻CD14⁺ DC precursors, and undifferentiated CD34⁺ cells) were collected.

Chemotaxis assay. Supernatants from Caco-2 cells cultured in complete DMEM (2% FCS), or rhCCL20 were added to 24 well plates and 5×10⁵ DCs to Transwell inserts (5 μm pores, Corning Inc.). Plates were incubated for 1.5 h at 37° C. Migrated cells were stained with FITC-labeled anti-CD1a mAb and PE-labelled anti-CD14 mAb and counted by flow cytometry. For neutralization, samples were incubated for 30 min at 37° C. with 10 μg/ml of goat anti-CCL20 Ab.

Flagellin purification for immunization of mice. FliC-producing S. typhimurium strain SIN22 (fljB5001::MudJ) and flagellin-deficient SIN41 (fliC5050::MudJ fljB5001::Mud-Cam) were obtained by phage P22 HT105/int-1 transduction using strains TH714 and TH2795 (gifts from K. Hughes), respectively, as donor and the wildtype strain ATCC14028 as recipient (43, 47). Flagellin was prepared from strain SIN22 grown for 16 h at 37° C. with agitation in Luria Bertani medium as described previously (48). Briefly, flagella were sheared from surface, pelleted by ultracentrifugation, and heated for 30 min at 65° C. to release flagellin monomers (5 mg/l culture). Flagellin was concentrated in PBS, filtered through 100 kD cut-off device, depleted of endotoxin activity using Detoxi-Gel Affinity Colums (Pierce), and stored at −20° C. Endotoxin contaminations were quantified using Limulus amebocyte lysate Pyrochrome assay (Cape Cod incorporated); in the four independent batches used in this study, endotoxin amounts were less than 20 pg per μg flagellin. Similar preparation from strain SIN41 without endotoxin depletion were performed to control contamination by microbial products unrelated to flagellin. When specified, flagellin was totally digested at 37° C. for 30 min with cell culture quality trypsin 0.05%/EDTA 0.02% solution (Biochrom AG) followed by 1 h inactivation at 70° C. Flagellin purity was assayed by SDS-PAGE analysis and immunoblot with flagellin-specific mouse polyclonal serum and peroxidase-conjugated anti-mouse IgG (Biorad). Flagellin-specific serum was obtained on day 35 from C57BL/6 immunized subcutaneously twice on days 0 and 26 with 40 μg flagellin+CFA and 20 μg flagellin+IFA, respectively.

Ovalbumin (OVA, Grade VII, Sigma) and hen egg lysozyme (HEL, Appligen) were also detoxified using polyrmixin column (<20 μg endotoxin per μg protein). Protein concentration was determined by the Bradford microassay (Biorad).

Antibodies and flow cytometry. FACS® staining analysis was performed using the following mAbs: anti-CD11c-FITC or -PE or -biotin (clone HL3), anti-MHC II-PE (clone 2G9), anti-B220-Cy5 or -CyChrome (clone RA3.6B2), anti-CD8α-CyChrome (clone 53.6.7), anti-CD4-CyChrome or -biotin (clone LT4), anti-CD80-biotin (clone 16-10A1), anti-CD86-biotin (clone GL-1), anti-CD40-biotin (clone 3/23) (PharMingen). Anti-F4/80-FITC or -biotin (clone F4/80) and anti-MHC II-biotin (clone 11.54.3) were purified and conjugated in the laboratory. Biotinylated C4H3 mAb that recognizes peptide HEL₄₆₋₆₁ of in the context of I-A^{k} (49) was a kind gift of Pr. R. Steinman (Yale University, USA). Biotinylated antibodies were revealed with streptavidin conjugated either to PE (Serotec), CyChrome (PharMingen) or allophycocyanin (Molecular Probes). Flow cytometry was performed using three or four colors FACSCalibur™ cytometer and analyzed using CELLQuest™ software (Becton Dickinson).

RT-PCR analysis of TLR5 mRNA. Total RNA was isolated and treated with DNase I (Quiagen). Reverse transcription (RT) was performed using Superscript II (Gibco BRL). For mouse BM or splenic cells, cDNA was amplified by the SYBR®-Green PCR assay using primers specific for TLR5 CGCACGGCTTTATCTTCTCC, GGCAAGGTTCAGCATCT TCAA and for 18S ribosomal RNA to standardize the total amount of 18S RNA, as described (47). The specificity of the PCR was checked by analyzing melting curves and sequencing. Relative mRNA levels (2^{ΔΔC}) were determined by comparing (i) the PCR cycle threshold (C) between cDNA of the gene of interest and of 18S rRNA (ΔC), (ii) ΔC values subtracted to ΔC value obtained for total splenocytes, which was chosen as an arbitrary reference (ΔΔC).

(For human cells, standard PCR was performed with 40 cycles (94° C. 45 sec, 53° C. 45 sec, 72° C. 1 min) using the following primers for human TLR5: AGTTCTCCCTTTTCATTGTATG and GAATCTGTTTTGGTCACTGTAT (259 bp), and human β-actin: TGACGGGGTCACCCACACTGTGCCCATCTA and CTAGAAGCATTGCGGTGGACGATGGAGGG 660 bp).

Detection of flagellin- and OVA-specific antibodies in serum of immunized animals. Serum was sampled and analyzed by ELISA as described (42). For IgG measurements, microplates (Maxisorp Nunc, Life Technologies) were coated with 100 ng flagellin or 1 □g OVA per well in PBS. Preimmune sera and sera from mock-immunised mice were used as negative controls. Detection of total IgG was performed with peroxidase-conjugated goat anti-mouse IgG (Biorad) and titres were expressed as reciprocal of the highest dilution that yielded an absorbency of 0.1. Absolute quantification of IgG1 and IgG2a titres was performed using reference serum and either rabbit anti-mouse IgG1 or sheep anti-mouse IgG2a conjugated to peroxidase (Serotec) or biotin-conjugated goat anti-mouse IgG1 and IgG2a (Caltag).

Preparation of mouse bone marrow derived dendritic cells. Bone marrow-derived DCs were cultivated from femoral and tibial bones of mice (44). Briefly, the bone marrow cells were depleted in RBC, plated at 2×10⁵ cells ml⁻¹ in culture-treated 6-well plates (Nunc) in the presence of 10 ng/ml of GM-CSF (Biosource) in complete RPMI 1640 medium containing 10% FCS (Myoclone superplus), 2 mM L-glutamine, 10 mM HEPES, 1 mM sodium pyruvate (Gibco BRL). Three days later, fresh medium supplemented with GM-CSF was added. On day 6, either whole cells or floating cells were stimulated as mentioned. The cell phenotype was analyzed by flow cytometry using antibodies specific for various surface markers.

To analyse TLR5 expression, bone marrow CD11c⁺ cells were isolated by positive selection using MACS magnetic beads coupled to CD11c antibodies (N418, Miltenyi Biotech). The purity of DCs was ˜90%.

Analysis of splenocytes and cytokine release. To analyse splenocytes and cytokine release in the serum, flagellin, S. typhimurium LPS (L-6511, Sigma) or phosphorothioate CpG oligonucleotide (TCCATGAC GTTCCTGATGCT, Eurogentec) were injected in the tail vein. Spleens were harvested for isolation of splenocytes and/or sera were collected at different time points depending on the experiment.

Preparation of cells from spleen of immunized mice. Splenic DCs were isolated as previously described (41). Briefly, small fragments of spleen were incubated with 0.5 mg/ml Collagenase D (Roche) and 40 μg/ml Dnase I (Roche) in RPMI 1640 medium supplemented with 5% FCS for 10 min at 37° C. After mechanical dissociation, the cells were extensively washed in PBS supplemented with 5% FCS and 5 mM EDTA and resuspended in cold isoosmotic Optiprep™ solution (Nycomed) containing 5 mM EDTA. Upon centrifugation, the low-density fraction (LDF) routinely contains 20-30% DCs compared to 1% in the spleen. To assess TLR5 expression, DC (CD11c⁺ F4/80^{-/low} B220⁻), B lymphocytes 03220⁺ CD11c⁻) and macrophages(F4/80^{high}) were isolated by FACS-sorting on a FACStar™ flow cytometer (Becton Dickinson) after staining with appropriate antibodies.

Cytokine specific ELISA. Cytokines (IL-12 p40, IL-12 p70 and TNF-α) were quantified in sera and culture supernatant by sandwich ELISA kits from Pharmingen according to the manufacturer's recommendations.

Preparation of human dendritic cells. For human DC preparation, progenitors were isolated from umbilical cord blood by positive selection using anti-CD34 mAb (Immu-133.3, Immunotech), goat anti-mouse IgG microbeads and MACS® (Miltenyi Biotec). CD34⁺ cells were grown in RPMI-1640, 10% FCS, 200 U/ml rhGM-CSF (Schering-Plough Research), 50 U/ml rhTNF□ (PeproTech Inc.) and 10 U/ml rhSCF (R&D Systems). After 7 days, the cells (30-50% CD1a⁺ DCs, 25-35% CD1a⁻CD14⁺ DC precursors, and undifferentiated CD34⁺ cells) were collected.

Adoptive transfer experiments. OVA-specific CD4⁺ T cells were isolated from spleen and lymph nodes of DO11.10 SCID mice using MACS CD4 beads (Miltenyi Biotech) with a purity >98%. The cells were stained with 5 μM carboxyfluorescein diacetate succininyl ester (CFSE, Molecular Probes) and 4-5×10⁶ cells were injected i.v. in BALB/c recipient mice. One day later, mice were immunized i.v. with either PBS, flagellin and/or OVA and the splenocytes from immunized animals were analyzed 72 hours later by flow cytometry. CFSE positive cells were then detected and counted among Vβ8⁺ (present in the transgenic TCR) CD4⁺ cells.

### Example 1

S. typhimurium induces expression of CCL20 gene in intestinal epithellal cells. The expression of the CCL20 chemokine gene in the human intestinal epithelial Caco-2 cell line grown on permeable filters in response to various stimuli by real time RT-PCR and by ELISA. In untreated cells about 1.8±1.0×10⁶ CCL20 copies per μg of total were detected which corresponds to ˜10 copies per cell. The concentration of CCL20 in the apical and basal medium never exceeded 0.5 ng/ml. These observations confirmed the constitutive CCL20 gene expression in Caco-2 cells reported by others (11).

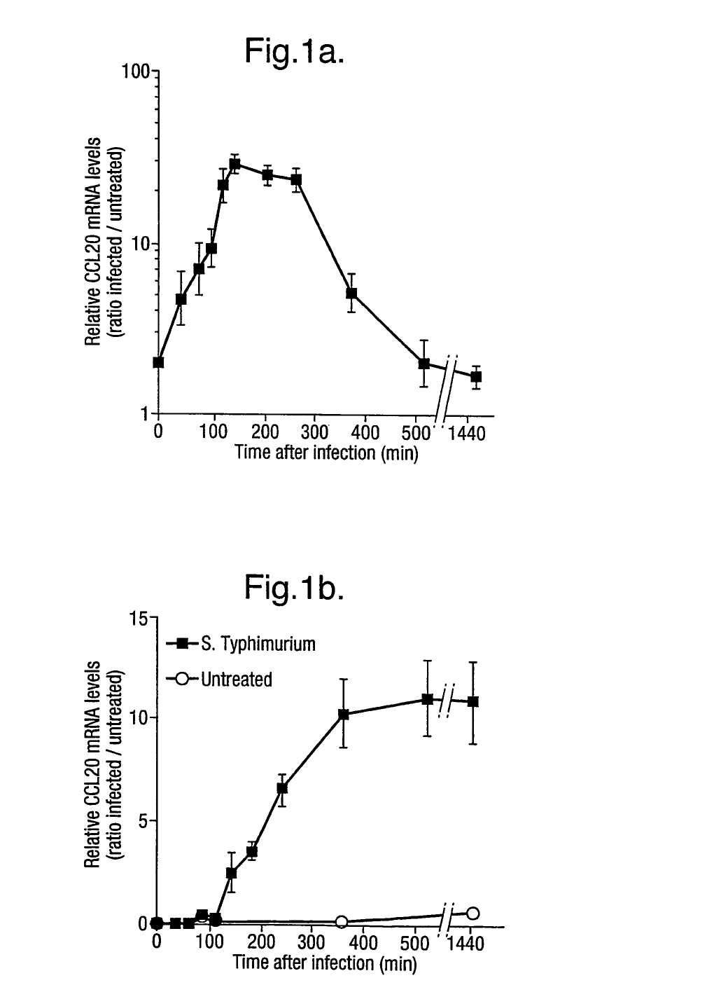

Apical exposure of Caco-2 cells to virulent S. typhimurium ATCC14028 resulted in efficient infection since 0.25% bacteria were internalized within 2 h (supplementary material). The transcription of CCL20 was maximally increased between 2 and 3.5 h after infection [15.2±6.9 fold (n=20)] (FIG. 1a). Under these conditions, IL-8 transcription was increased 27.6±7.8 fold as reported previously (1, 15). CCL20 secretion increased significantly 2 h after infection and reached a plateau at 6 h (FIG. 1b). CCL20 secretion was partially polarized since 20 h after infection 64.8±6.9 % (n=7) were recovered in the basal compartment.

### Example 2

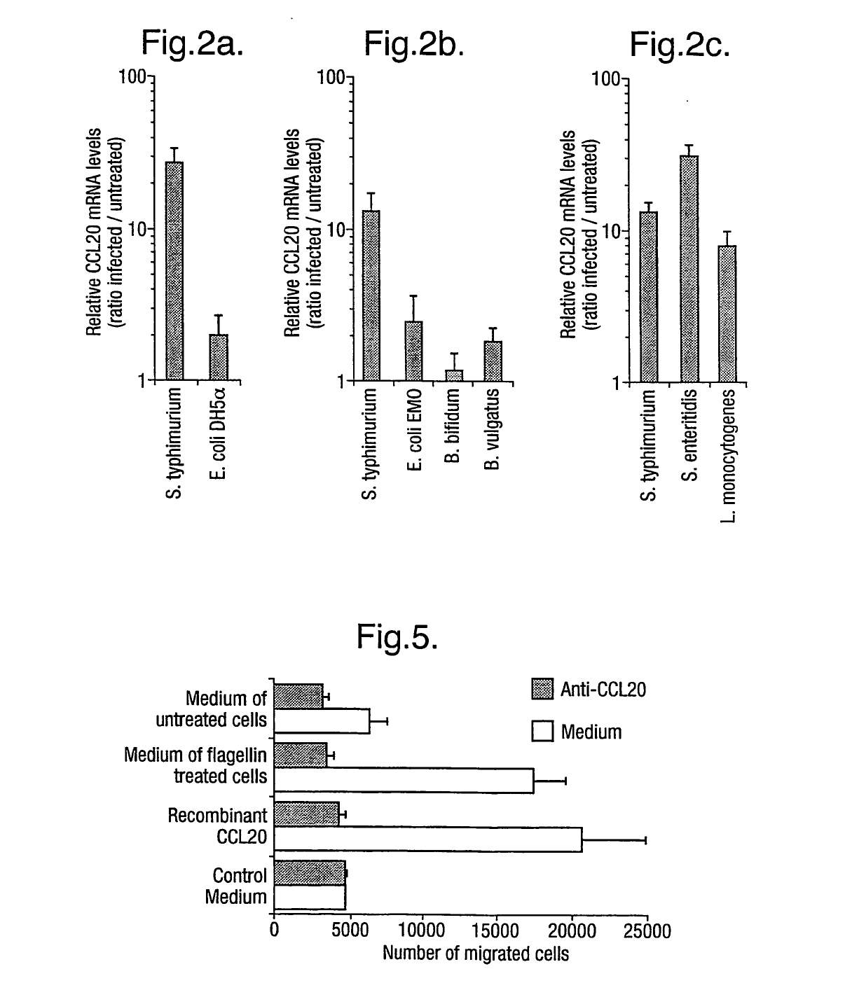

The CCL20 response is induced by pathogens. The specificity of CCL20 induction was analyzed in response to various bacteria encountered in the gut. The E. coli strain DH5-α and the commensal bacteria E. coli EMO, B. bifidum, or B. vulgatus were unable to induce CCL20 expression (FIGS. 2a and b). In contrast, pathogenic bacteria including S. enteritidis and L. monocytogenes activated CCL20 transcription as efficiently as S. typhimurium (FIG. 2c).

### Example 3

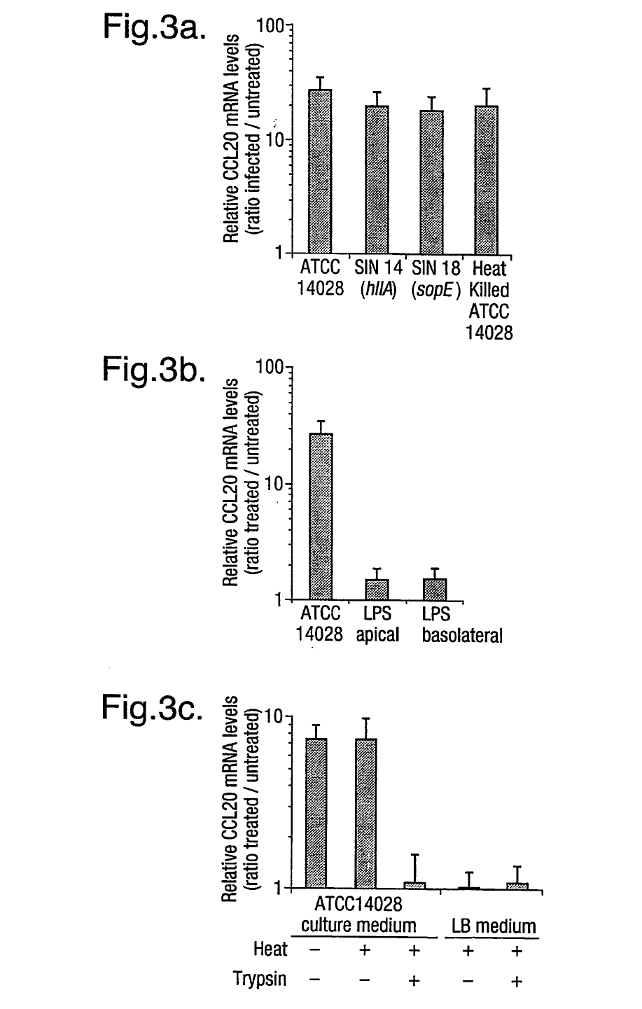

CCL20 induction does not require epithelial cell invasion. Invasion of epithelial cell is dependent on a type III secretion system encoded by Salmonella pathogenicity island 1 (SPI-1) that injects toxins, such as SopE, in the cytoplasm of epithelial cells (13, 22). These toxins induce membrane ruffles resulting in bacterial internalization and disturb signalling pathways. Inactivation of the hilA gene that encodes an activator of SPI-1 genes impairs invasion. The S. typhimurium hilA mutant SIN14 and the sopE-inactivated strain SIN18 were found as efficient as ATCC14028 to induce CCL20 expression in epithelial Caco-2 cells (FIG. 3a). CCL20 induction by heat-killed and live bacteria was not significantly different (FIG. 3a), thus ruling out a role of bacterial invasion. Therefore, our experiments indicated that CCL20 stimulation does not require epithelial cell invasion nor the injection of SopE toxin.

### Example 4

CCL20-inducing factor is a heat stable secreted protein. S. typhimurium supernatant strongly induced CCL20 expression when applied apically on epithelial cells (FIG. 3c). LPS is a heat-resistant molecule of outer membrane from Gram negative bacteria involved in cell signalling. Apical or basal treatment of Caco-2 cells with commercial S. typhimuriun LPS or LPS purified from ATCC14028 did not activate CCL20 gene transcription (FIG. 3b). Thus, LPS per se is not the induction factor for CCL20 stimulation.

As observed with whole bacteria, heat treatment did not abolish the supernatant activity (FIG. 3c). Trypsin digestion of the supernatant, however, totally abrogated CCL20 induction. Altogether, these experiments indicated that the CCL20-specific induction factor is a heat-stable secreted protein.

### Example 5

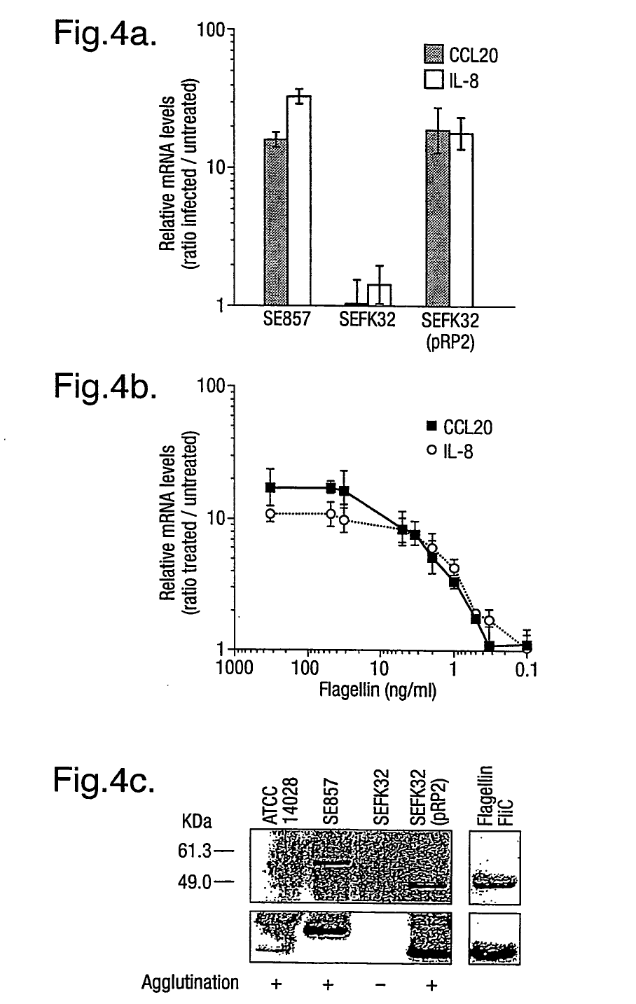

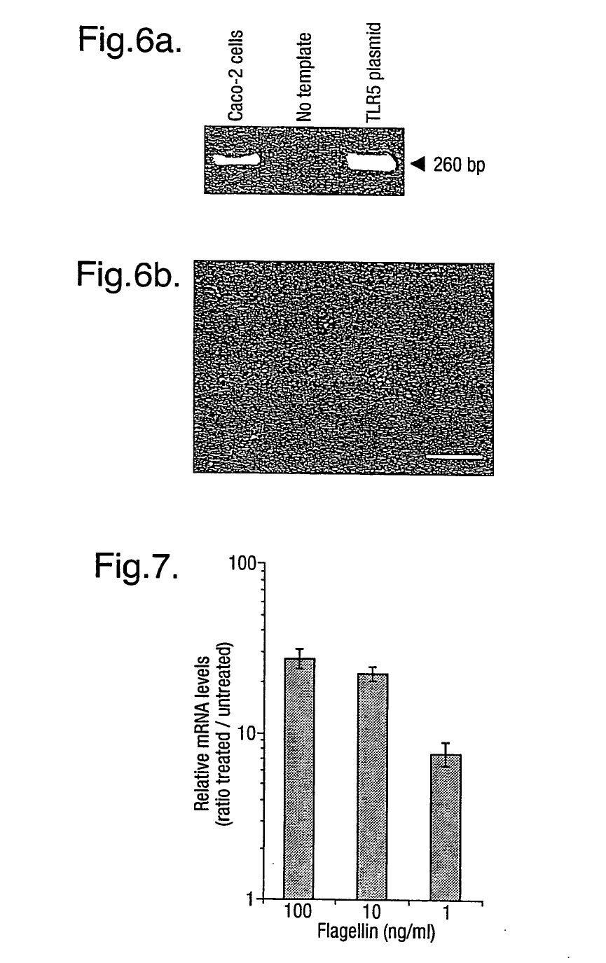

Flagellin is the CCL20 induction factor. Flagellin, the subunit constituting the flagellar filament, is the major protein recovered from S. typhimurium or S. enteritidis supernatants (FIG. 4c) (23). S. typhimurium produces two 52 KDa flagellins: FliC or FljB whereas S. enteritidis produces a single 56 KDa flagellin FliC. The fliC-deleted S. enteritidis SEFK32 was unable to induce CCL20 gene expression in contrast to the parental strain SE857 (FIG. 4a). Complementation of fliC mutant with fliC gene from S. typhimurium fully restored CCL20 induction. In addition, S. typhimurium mutants SIN20 or SIN22 producing either FljB or FliC stimulated CCL20 transcription to similar levels as wild-type bacteria (data not shown). Finally, purified S. typhimurium FliC flagellin activated CCL20 transcription in Caco-2 cells (ED₅₀˜20 pM and FIG. 4b). Similar results were obtained using the T-84 epithelial cell line (see Table). As recently reported (21, 24), flagellin was found to induce transcription of the IL-8 gene in Caco-2 cells (FIG. 4a-b). Our experiments demonstrated that flagellin is required for the induction of CCL20 and IL-8 gene expression in epithelial intestinal cells.

### Example 6

Medium from flagellin-treated cells induces migration of immature DCs. Human immature DCs were able to migrate in response to rhCCL20 (FIG. 5). The migration was inhibited by incubation with CCL20-specific antibodies. Low migration of DCs was observed with basal medium from untreated cells, probably reflecting the constitutive secretion of CCL20 by Caco-2 cells. The basal medium from flagellin-treated Caco-2 monolayers was as chemotactic as rhCCL20 at equivalent concentrations. Moreover, incubation of medium with CCL20-specific mAb fully abrogated chemotaxis. In conclusion, the migration of immature DCs medium from flagellin-stimulated Caco-2 is specifically dependent on CCL20 activity.

### Example 7

Flagellin sequences 190 and 354-494 are required for epithelial cell signalling. Stable transfectants of Caco-2 cells (human intestinal epithelial cells) with plasmid containing ccl20 promoter linked to firefly luciferase reporter gene were treated with various concentrations of flagellin, flagellin fragments or genetically engineered flagellin mutants in 96 wells microplates. (Truncated flagellin molecules were generated by genetic engineering of flicC encoding plasmid or by trypsin digestion of flicC flagellin from S. typhimurium.) After 18 h incubation, cells were lysed and assayed for luciferase activity using Steady-Glo reagent (Promega). The fold increase in ccl20 gene transcription was determined as ratio of luminescence of sample on luminescence of cells treated with PBS.

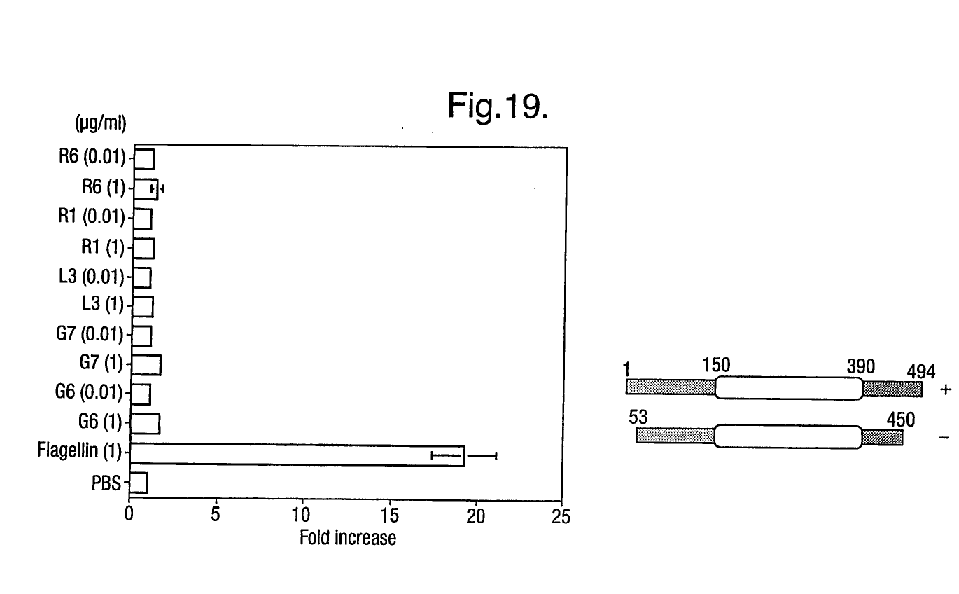

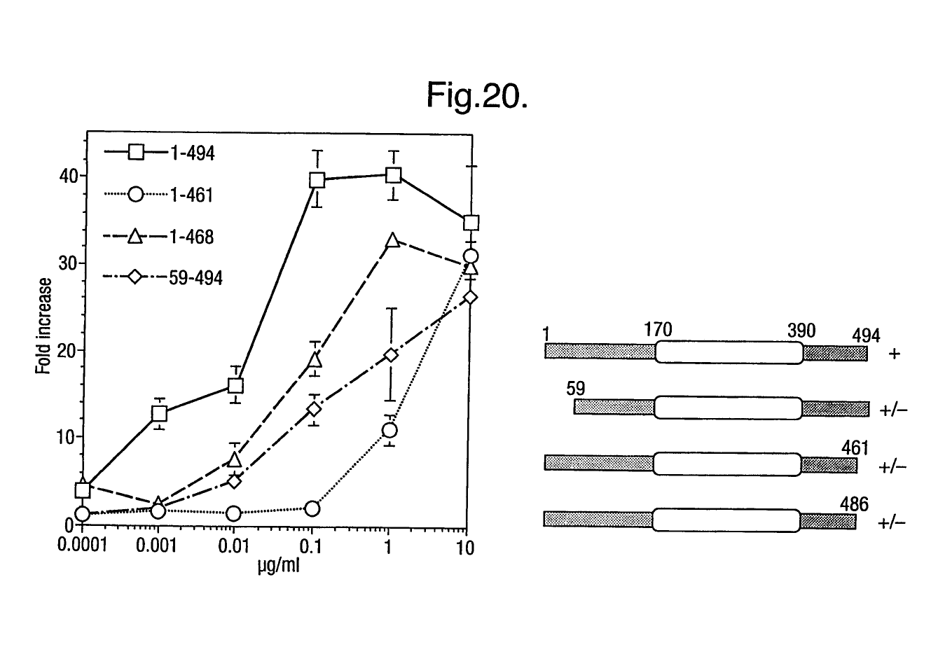

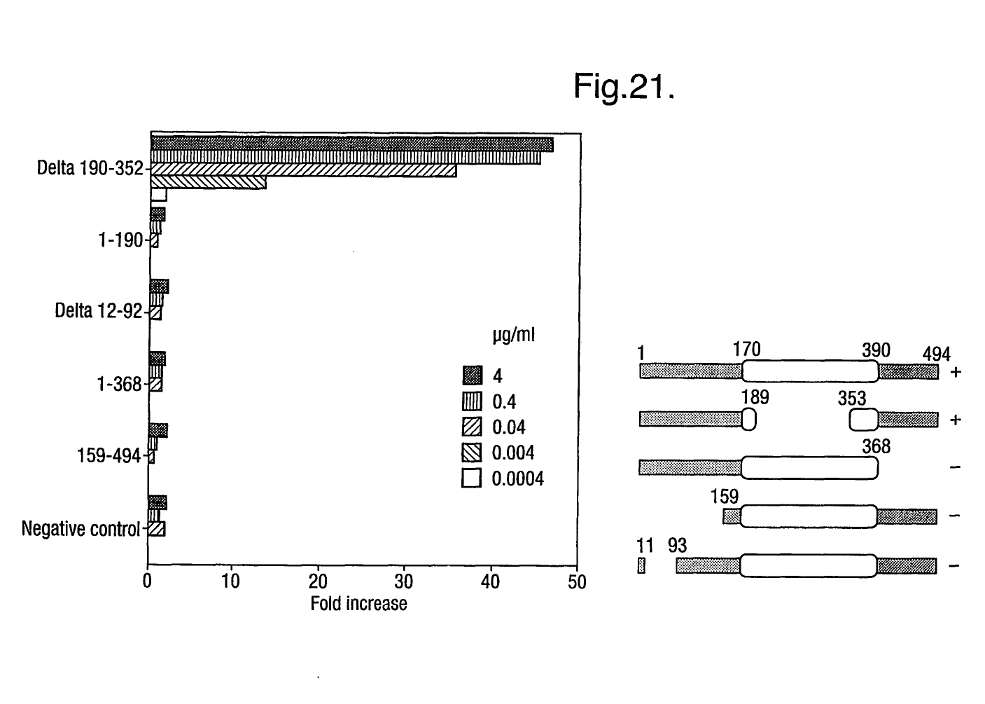

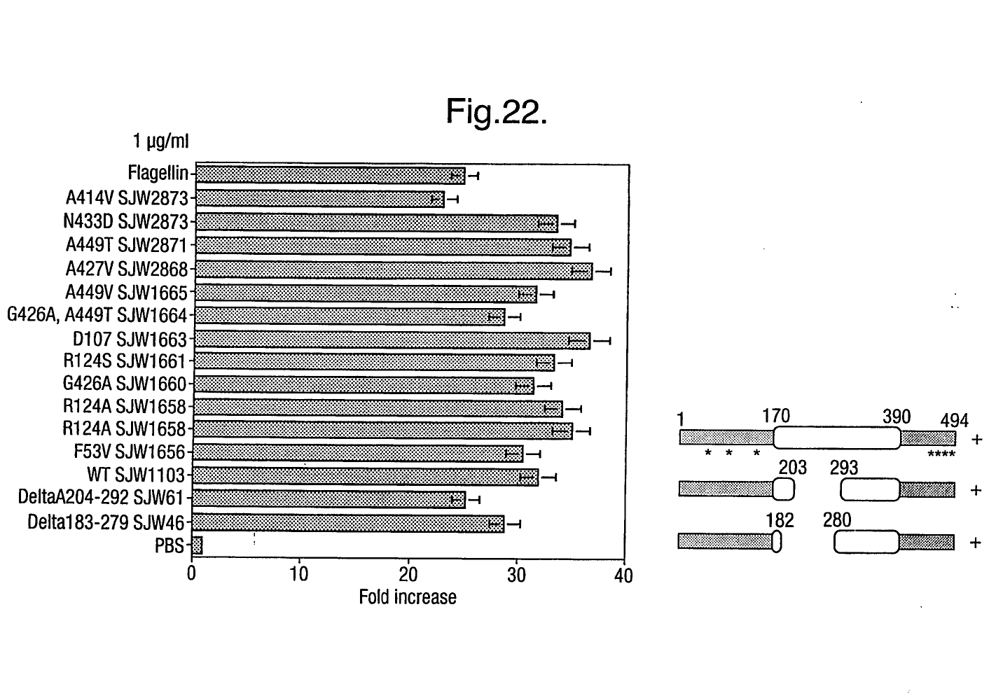

Results shown in FIGS. 19, 20 and 21 demonstrate that the amino and carboxy terminal regions are required for cell signalling. The central region (between residues 191 and 353), which is variable among flagellins from various Salmonella serotypes and from various bacterial species, does not play a role in signalling. Therefore, the essential region seems to be confined in two regions: residues from 1 to 190 and residues from 354 to 446.

### Example 8

Stimulation of antigen-specific IN-γ producing CD8+ lymphocytes by subcutaneously administered flagellin and MHC class I-restricted peptide.

IFN-γ ELISPOT are used to detemine the frequency of lymphocytes that produces IFNγ in a lymphoid organ. Using an ovalbumin MHC class I restricted-peptide the inventors have now shown an increase of number of lymphocytes responding to the peptide when the peptide is administered subcutaneously (base of the tail) in presence of flagellin. As negative control, peptide alone was injected. As positive control, peptide was injected with an universal helper peptide derived from tetanus toxin and incomplete Freund's adjuvant (a mineral oil): this is known to trigger a strong CD8 response.

Protocol is as follows: IFN-γ ELISPOT
Keep sterile until adding secondary INF-γ antibody
Few days before, split APC cells to have enough cells for the experiment
Make at least quadriplicate for each conditions

DAY-1

1—Coat overnight at RT the Mulltiscreen microplates with 75 μl/well of capture antibody (34.1) diluted at 6 μg/ml of PBS->44 μl in 7.3 ml per plate

2—Design the plates on paper

DAY 0

3—Collect tissues and effector cells on mice
The effector cells can be obtained from spleen or any other lymphoid tissue. Homogenize or digest the lymph nodes or the spleen and lyse the red blood cells.
   wash the plate 3 times in RPMI-10 and let saturate with 100 μl/well in the incubator (at least 1 hour).

4 Count effector cells
Wash effector cells in RPMI-10 and resuspend in 10 ml.
count the cells and prepare tubes with appropriate cell concentration and volume in order to dispatch 100 μl/well.

Usually: for splenocytes or LN cells, count 1.10⁶ cells for 600 μl as first point. Then make serial dilution 1:10. Each point is done in triplicates.

5 Distribute cells
Discard complete RPMI off the plates
add 100 μl/well of effector cells+peptide 20-100 μM in 100 μl RPMI-10/well
incubate 15-24 hours at 37° C., 5 % CO2.

DAY +1

6 Develop the Elispot
Wash once with water
Wash 4 times with PBS-Tween 0.05%
Incubate 2 h at 37° C. (or overnight at 4° C.) with 75 μl/well of the detection antibody (35.1) diluted at 2 μg/ml of PBS-T0.05->30 μl in 7.3 ml per plate
Wash 4 times with PBS-T0.05
Incubate for 1 h at RT with 100 μl/well of Alkaline Phosphatase(AP)-Extravidin number 118 (diluted 1:5000 in PBS-T)
Wash 4 times in PBS-T
Wash 2 times in PBS, no tween
Add 100 μl/well of AP reagent and incubate at RT in the dark for 15-20 min (blue spots).
Stop the reaction by washing extensively with tap water and let dry.
Materials:
Mulltiscreen microplates 96 wells (HA sterile plates 0.45 μm, Millipore n°MAHAS4510)
Capture antibody: rat anti-mouse IFN-g R4-6A2 (Pharmingen n°8181D).
Detection antibody: biotin-conjugated rat anti-mouse IFN-g XMG1.2 (Pharmingen n°18182D)
Extravidin-AP=Extravidin conjugated to Alkaline Phosphatase (Sigma, E26-36, n°118 at 4° C.)
AP reagent:
Buffer: -Tris-HCl pH 9.5 100 Mm
NaCl 100 mM
MgCl₂ 50 mM
For 10 ml of buffer, add

67 μl of Nitroblue tetrazolium chloride (NIBT) at 50 mg/ml in 100% Methanol (Sigma N-5514)-> final concentration: 0.33 mg/ml

20 μl of 5-Bromo-4-chloro-3 indolylphosphate (BCIP) p-toluidine salt (Sigma B-8503) at 50 mg/ml in 100% dimethyl-Formamide (DMEF)->final concentration: 0.1 mg/ml

### Example 9

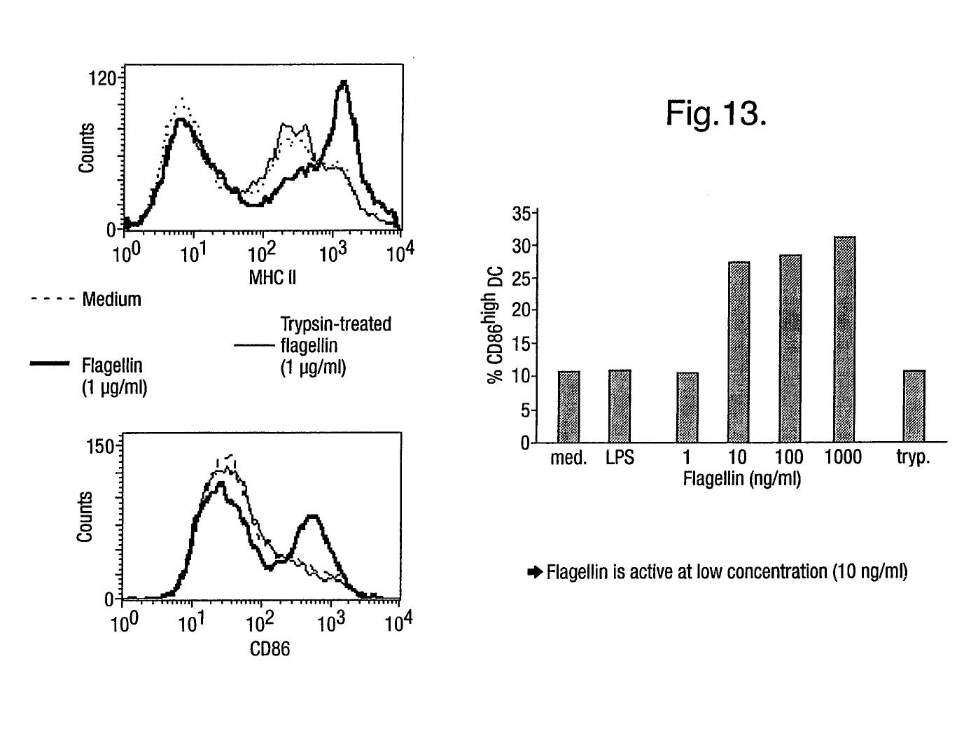

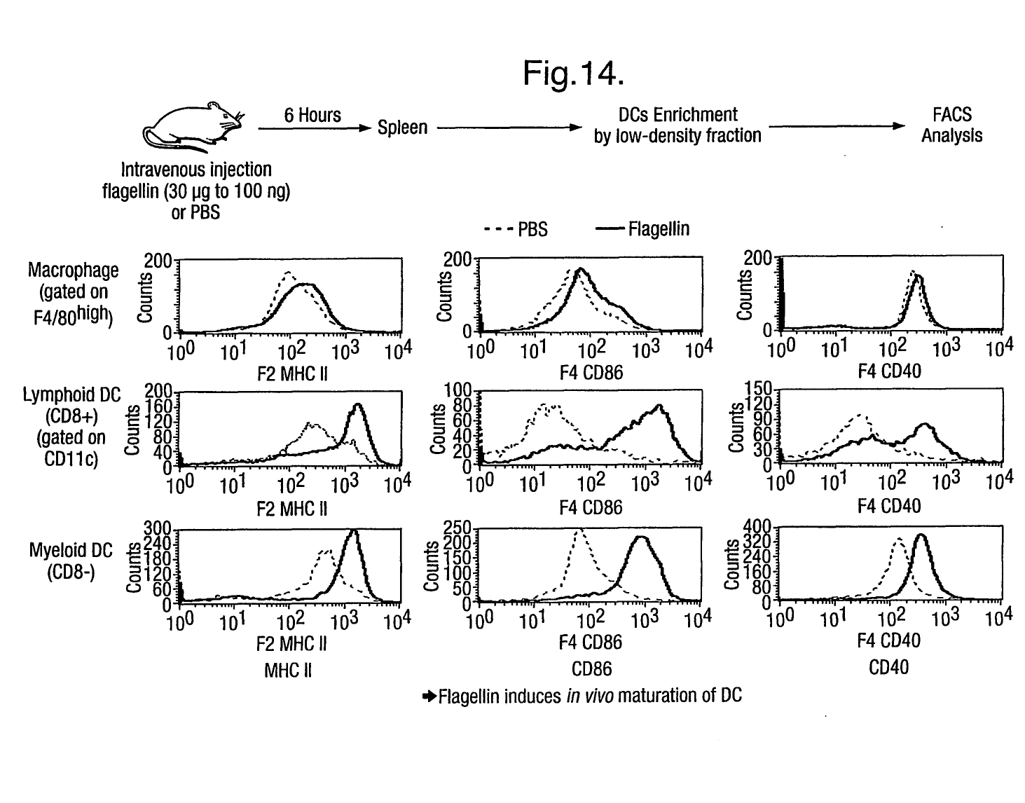

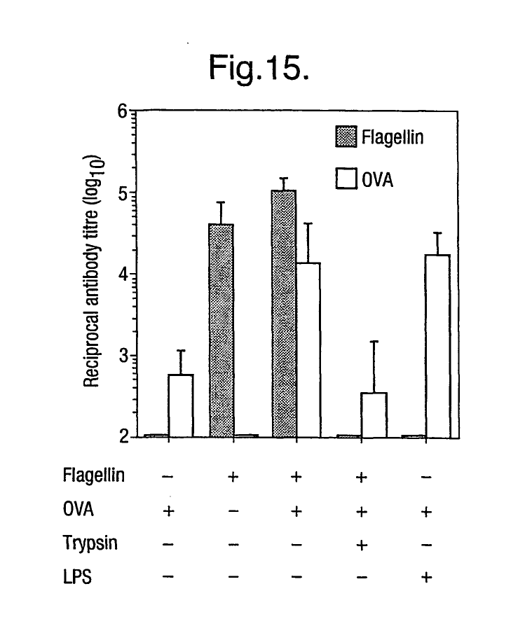

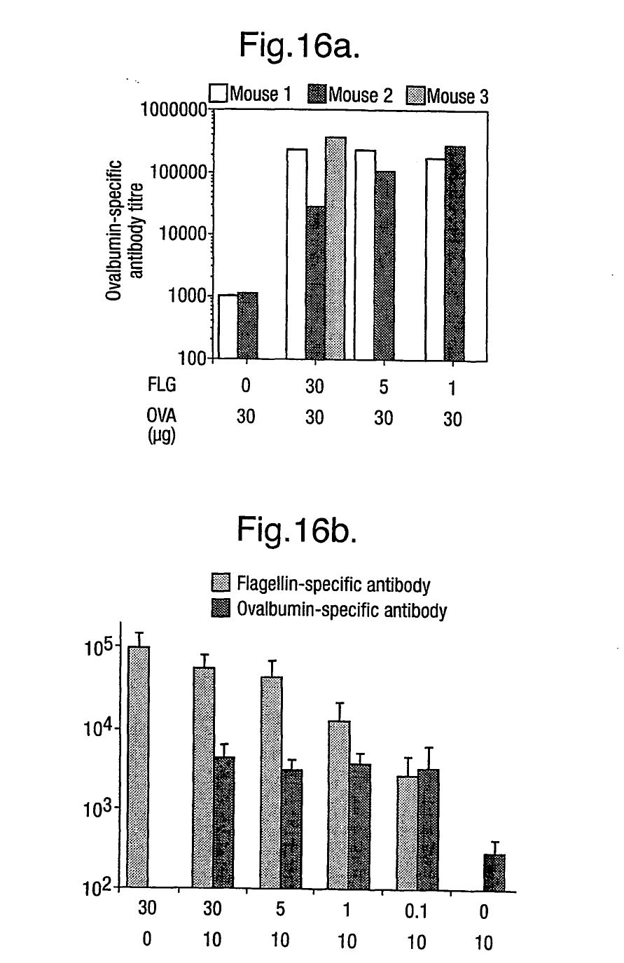

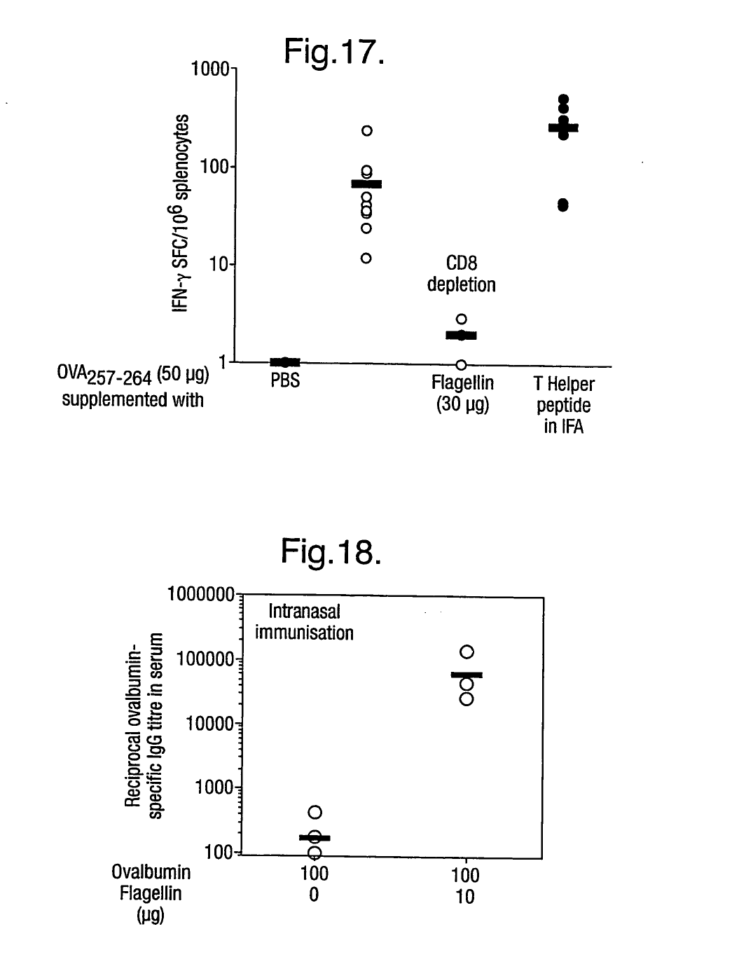

Dendritic cells are specifically activated by flagellin. Mice were injected intravenously (tail vein) with PBS, flagellin or LPS (S. typhimurium). 6 hours post injection the spleens were harvested and splenocytes isolated. The isolated cells were analysed by flow cytometry. As shown in FIGS. 13A and 13B, dendritic cells are specifically activated by flagellin.

### Example 10

Flagellin is a systemic adjuvant. Mice were immunized subcutaneously (base of tail) on days 0 and 21 with PBS, flagellin, trypsin treated flagellin or LPS with ovalbumin. Serum was collected on days 28/35 and the serum antibody response measured using ELISA.

### Example 11

Systemic administration of flagellin potentiates the serum antibody response. Mice (inbred BALB/c and outbred NMRI) were immunized by subcutaneous route with flagellin (0.1 to 30 □g) and/or OVA (10-100 □g) formulated in 200 □l endotoxin-free PBS. Injections were performed on day 0 and day 21 days. Serum was sampled 2 and 5 weeks later and antibody response specific for flagellin or OVA was analysed by ELISA.

### Example 12

Flagellin induces maturation of dendritic cells in-vitro. Bone marrow-derived DCs were cultivated from femoral and tibial bones of mice as described above (4). Briefly, the bone marrow cells were depleted in RBC, plated at 2×10⁵ cells ml⁻¹ in culture-treated 6-well plates (Nunc) in the presence of 10 ng/ml of GM-CSF (Biosource) in complete RPMI 1640 medium containing 10% FCS (Myoclone superplus), 2 mM L-glutamine, 10 mM HEPES, 1 mM sodium pyruvate (Gibco BRL). Three days later, fresh medium supplemented with GM-CSF was added. On day 6, either whole cells or floating cells were stimulated as mentioned.

The cell phenotype was analyzed by flow cytometry using antibodies specific for various surface markers.

### REFERENCES

1. Eckmann, L., Kagnoff, M. F. & Fierer, J. (1993) Infect. Immun. 61, 4569-74.
2. McCormick, B. A., Miller, S. I., Carnes, D. & Madara, J. L. (1995) Infect. Immun. 63,2302-9.
3. Finlay, B. B. & Falkow, S. (1997) Microbiol. Mol. Biol. Rev. 61, 136-69.
4. Dieu, M. C., Vanbervliet, B., Vicari, A., Bridon, J. M., Oldham, E., Ait-Yahia, S., Briere, F., Zlotnik, A., Lebecque, S. & Caux, C. (1998) J. Exp. Med. 188, 373-386.
5. Charbonnier, A. S., Kohrgruber, N., Kriehuber, E., Stingl, G., Rot, A. & Maurer, D. (1999) J. Exp. Med. 190, 1755-68.
6. Iwasaki, A. & Kelsall, B. L. (2000) J. Exp. Med. 191, 1381-94.
7. Cook, D. N., Prosser, D. M., Forster, R., Zhang, J., Kuklin, N. A., Abbondanzo, S. J., Niu, X. D., Chen, S. C., Manfra, D. J., Wiekowski, M. T., Sullivan, L. M., Smith, S. R., Greenberg, H. B., Narula, S. K., Lipp, M. & Lira, S. A. (2000) Immunity 12, 495-503.
8. Tanaka, Y., Imai, T., Baba, M., Ishikawa, I., Uehira, M., Nomiyama, H. & Yoshie, O. (1999) Eur. J. Immunol. 29, 633-42.
9. Banchereau, J., Briere, F., Caux, C., Davoust, J., Lebecque, S., Liu, Y. J., Pulendran, B. & Palucka, K. (2000) Annu. Rev. Immunol. 18, 767-811.
10. Dieu-Nosjean, M. C., Massacrier, C., Homey, B., Vanbervliet, B., Pin, J. J., Vicari, A., Lebecque, S., Dezutter-Darnbuyant, C., Schmitt, D., Zlotnik, A. & Caux, C. (2000) J. Exp. Med. 192, 705-18.
11. Izadpanah, A., Dwinell, M. B., Ecknann, L., Varki, N. M. & Kagnoff, M. F. (2001) Am. J. Physiol. 280, G710-G719.
12. Kraehenbuhl, J. P. & Neutra, M. R. (2000) Annu. Rev. Cell Dev. Biol. 16, 301-332.
13. Hueck, C. J. (1998) Microbiol. Mol. Biol. Rev. 62, 379-433.
14. Worton, K. J., Candy, D. C., Wallis, T. S., Clarke, G. J., Osborne, M. P., Haddon, S. J. & Stephen, J. (1989) J. Med. Microbiol. 29, 283-94.
15. McCormick, B. A., Colgan, S. P., Delp-Archer, C., Miller, S. L & Madara, J. L. (1993) J. Cell Biol. 123, 895-907.
16. Yang, S. K., Eckmann, L., Panja, A. & Kagnoff, M. F. (1997) Gastroenterol. 113, 1214-23.
17. Gewirtz, A. T., Siber, A. M., Madara, J. L. & McCormick, B. A. (1999) Infect. Immun. 67, 608-617.
18. Elewaut, D., DiDonato, J. A., Kim, J. M., Truong, F., Eckmann, L. & Kagnoff, M. F. (1999) J. Immunol. 163, 1457-66.
19. Lee, C. A. & Falkow, S. (1990) Proc. Natl. Acad. Sci. USA 87, 4304-8.
20. Slauch, J. M., Mahan, M. J., Michetti, P., Neutra, M. R. & Mekalanos, J. J. (1995) Infect. Immun. 63, 437-41.
21. Steiner, T. S., Nataro, J. P., Poteet-Smith, C. E., Smith, J. A. & Guerrant, R. L. (2000) J. Clin. Invest. 105, 1769-77.
22. Hardt, W. D., Chen, L. M., Schuebel, K. E., Bustelo, X. R. & Galan, J. E. (1998) Cell 93, 815-26.
23. Komoriya, K., Shibano, N., Higano, T., Azuma, N., Yamaguchi, S. & Aizawa, S. I. (1999) Mol. Microbiol. 34, 767-79.
24. Gewirtz, A. T., Simon, P. O., Schmitt, C. K., Taylor, L. J., Hagedorn, C. H., O'Brien, A. D., Neish, A. S. & Madara, J. L. (2001) J. Clin. Invest. 107, 99-109.
25. Winstanley, C. & Morgan, J. A. (1997) Microbiol. 143, 3071-84.
26. Ciacci-Woolwine, F., Blomfield, I. C., Richardson, S. H. & Mizel, S. B. (1998) Infect. Immun. 66, 1127-34.
27. Eaves-Pyles, T., Murthy, K., Liaudet, L., Virag, L., Ross, G., Soriano, F. G., Szabo, C. & Salzman, A. L. (2000) J. Immunol. 166, 1248-1260.
28. Hayashi, F., Smith, K. D., Ozinsky, A., Hawn, T. R., Yi, E. C., Goodlett, D. R., Eng, J. K., Akira, S., Underhill, D. M. & Aderem, A. (2001) Nature 410, 1099-1103.
29. Krutzik, S. R., Sieling, P. A. & Modlin, R. L. (2001) Curr. Op. Immunol. 13, 104-108.
30. Cario, E. & Podolsky, D. K. (2000) Infect. Immun. 68, 7010-7017.
31. Hedlund, M., Frendeus, B., Wachtler, C., Hang, L., Fischer, H. & Svanborg, C. (2001) Mol. Microbiol. 39, 542-552.
32. Schultsz, C., Van Den Berg, F. M., Ten Kate, F. W., Tytgat, G. N. & Dankert, J. (1999) Gastroenterol. 117, 1089-97.
33. Neish, A. S., Gewirtz, A. T., Zeng, H., Young, A. N., Hobert, M. E., Karmali, V., Rao, A. S. & Madara, J. L. (2000) Science 289, 1560-1563.
34. Garside, P., Mowat, A. M. & Khoruts, A. (1999) Gut 44, 137-42.
35. Liao, F., Rabin, R. L., Smith, C. S., Sharma, G., Nutman, T. B. & Farber, J. M. (1999) J. Immunol. 162, 186-94.
36. Hopkins, S. A., Niedergang, F., Corthesy-Theulaz, L. E. & Kraehenbuhl, J. P. (2000) Cell. Microbiol. 2, 59-68.
37. Niedergang, F., Sirard, J. C., Blanc, C. T. & Kraehenbuhl, J. P. (2000) Proc. Natl. Acad. Sci. USA 97, 14650-14655.
38. Pron, B., Boumaila, C., 3aubert, F., Berche, P., Milon, G., Geissmann, F. & Gaillard, J. L. (2001) Cell. Microbiol. 3, 331-340.
39. Rescigno, M., Urbano, M., Valzasina, B., Francolini, M., Rotta, G., Bonasio, R., Granucci, F., Kraehenbuhl, J. P. & Ricciardi-Castagnoli, P. (2001) Nat. Immunol. 2, 361-7.
41. Anjuere, F., P. Martin, I. Ferrero, M. L. Fraga, G. M. del Hoyo, N. Wright, and C. Ardavin. 1999. Definition of dendritic cell subpopulations present in the spleen, Peyer's patches, lymph nodes, and skin of the mouse. Blood 93(2):590-598.
42. Coste, A., J. C. Sirard, K. Johansen, J. Cohen, and J. P. Kraehenbuhl. 2000. Nasal immunization of mice with virus-like particles protects offspring against rotavirus diarrhea. Journal of Virology 74(19):8966-8971.
43. Gillen, K. L., and K. T. Hughes. 1993. Transcription from two promoters and autoregulation contribute to the control of expression of the Salmonella typhimurium flagellar regulatory gene flgM. J Bacteriol 175(21):7006-7015.
44. Lutz, M. B., N. Kukutsch, A. L. Ogilvie, S. Rossner, F. Koch, N. Romani, and G. Schuler. 1999. An advanced culture method for generating large quantities of highly pure dendritic cells from mouse bone marrow. J Immunol Methods 223(1):77-92.
45. Murphy, K. M., A. B. Heimberger, and D. Y. Loh. 1990. Induction by antigen of intrathymic apoptosis of CD4+CD8+TCRlo thymocytes in vivo. Science 250(4988):1720-1723.
46. Poltorak, A., X. He, I. Smirnova, M. Y. Liu, C. V. Huffel, X. Du, D. Birdwell, E. Alejos, M. Silva, C. Galanos, M. Freudenberg, P. Ricciardi-Castagnoli, B. Layton, and B. Beutler. 1998. Defective LPS signaling in C3H/HeJ and C57BL/10ScCr mice: mutations in Tlr4 gene. Science 282(5396):2085-2088.
47. Sierro, F., B. Dubois, A. Coste, D. Kaiserlian, J. P. Kraehenbuhl, and J. C. Sirard. 2001. Flagellin stimulation of intestinal epithelial cells triggers CCL20-mediated migration of dendritic cells. Proc Natl Acad Sci USA 98(24):13722-13727.
48. Steiner, T. S., A. A. Lima, J. P. Nataro, and R. L. Guerrant. 1998. Enteroaggregative Escherichia coli produce intestinal inflammation and growth impairment and cause interleukin-8 release from intestinal epithelial cells. Journal of Infectious Diseases 177(1):88-96.
49. Zhong, G., C. Reis e Sousa, and R. N. Germain. 1997. Production, specificity, and functionality of monoclonal antibodies to specific peptide-major histocompatibility complex class II complexes formed by processing of exogenous protein. Proceedings of the National Academy of Sciences of the United States of America 94(25):13856-13861.

1. A method of inducing an adaptive immune response in a patient to a target antigen comprising administering to said patient a flagellin protein, or a peptide fragment thereof, in an amount effective to induce said response.

2. A method according to claim 1 wherein the flagellin or peptide fragment thereof is capable of directly inducing the dendritic cell adaptive immune response.

3. A method according to claim 1 wherein dendritic cell maturation is increased.

4. A method according to claim 1 wherein the flagellin or peptide fragment thereof is administered via the mucosal route.

5. A method according to claim 1 wherein the flagellin or peptide fragment thereof is administered orally or intranasally.

6. A method according to claim 1 wherein the flagellin protein includes at least one of the conserved regions of the N terminal sequence and the C terminal sequence of flagellin.

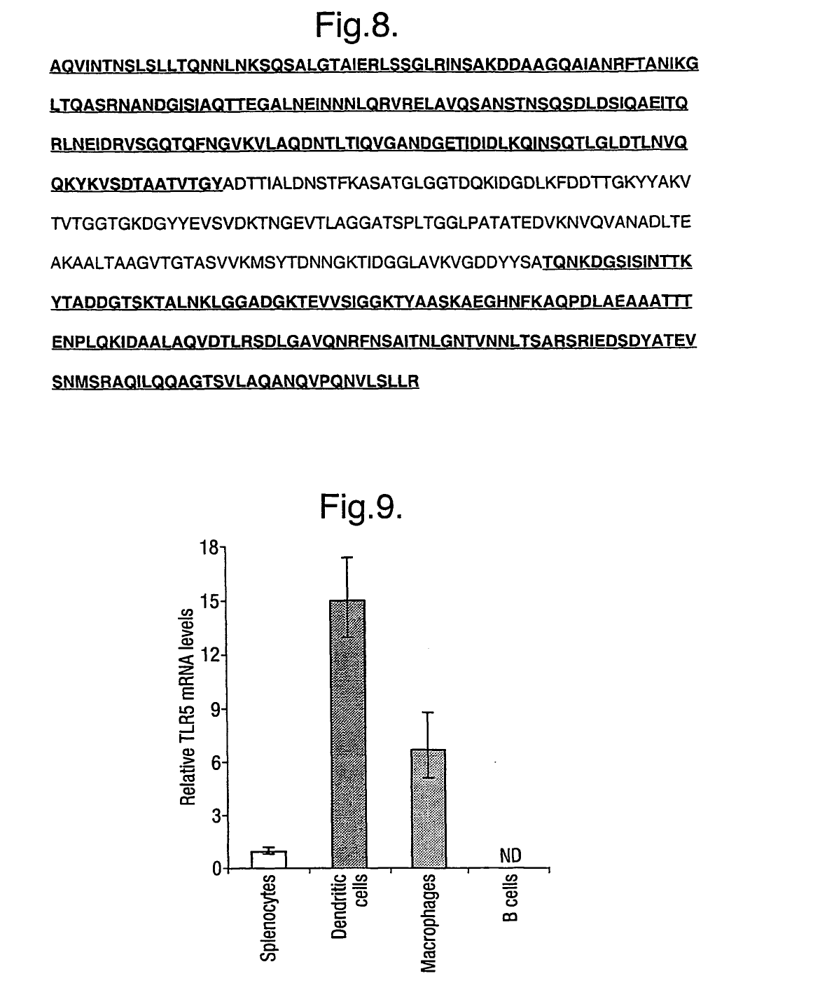

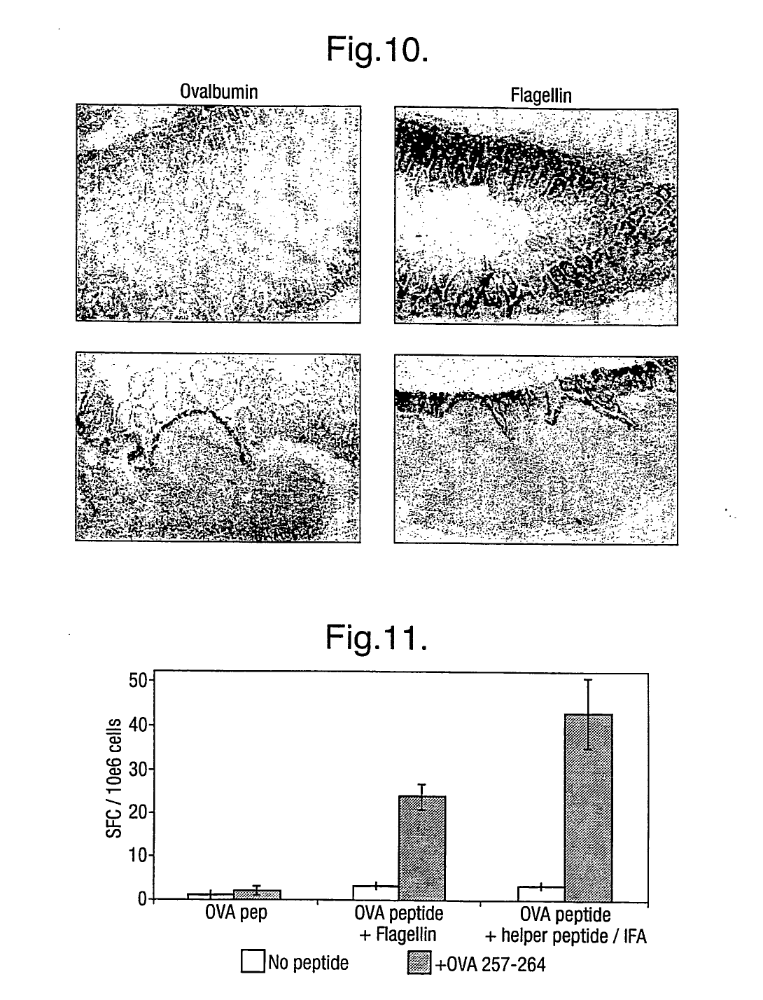

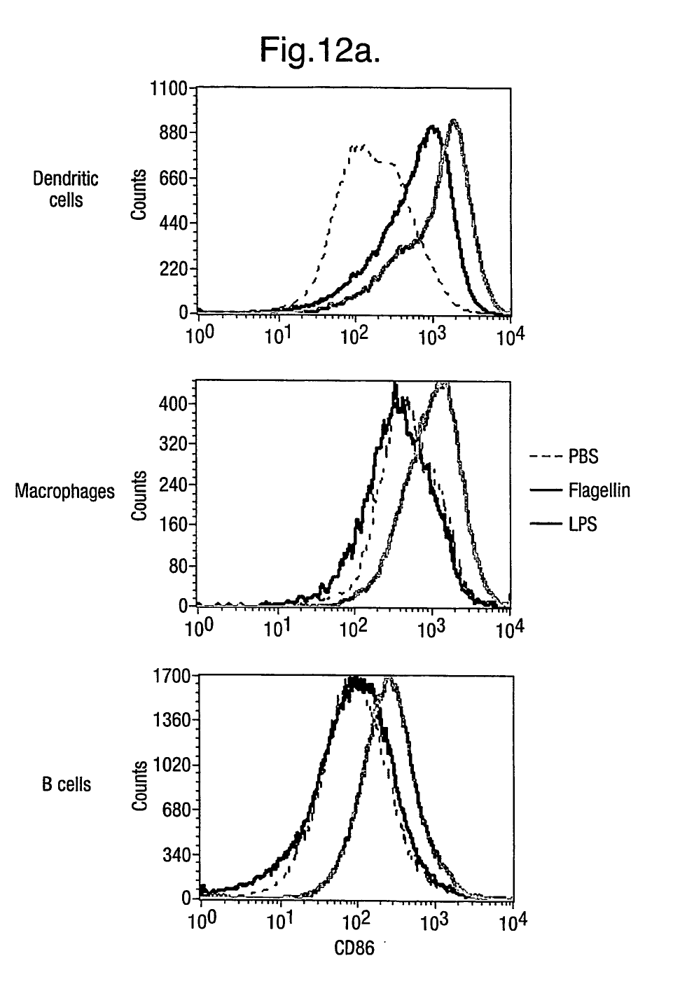

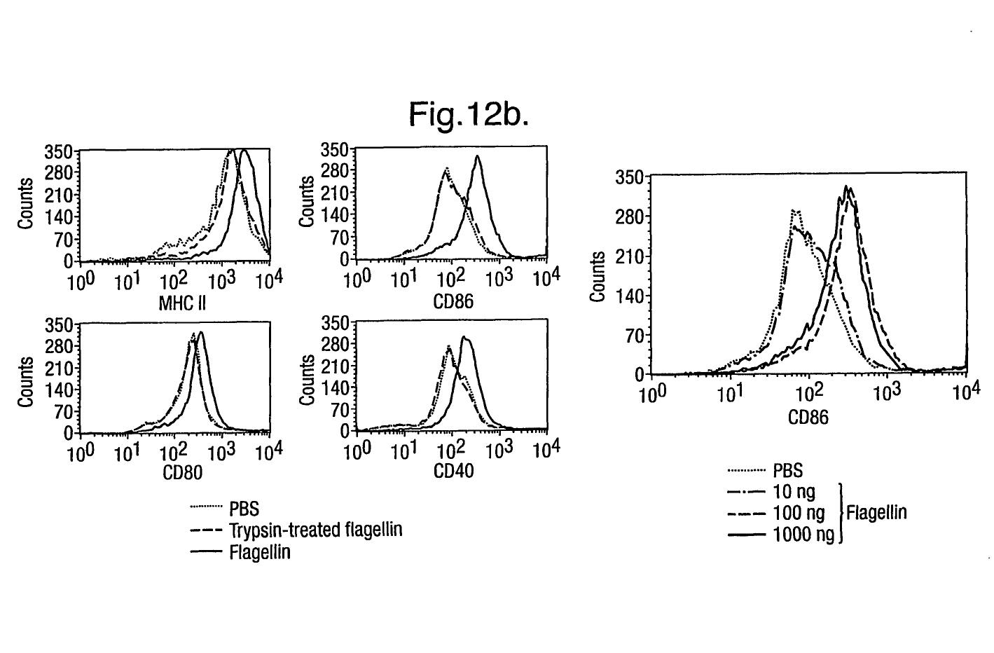

7. A method according to claim 1 wherein the flagellin protein includes at least one of the conserved regions of residues 1-190 and 354-494 of S. typhimurium as shown underlined in FIG. 8 herein.

8. A method as claimed in claim 1 wherein the flagellin protein or peptide fragment and the target antigen are co-administered.

9. Use of a flagellin protein or peptide fragment thereof in the manufacture of a medicament for the induction of an adaptive immune response.

10. Use as claimed in claim 9 characterised in that the medicament is for inducing recruitment of immature dendritic cells in mucosal vaccination such as to induce an adaptive immune response.

11. Use as claimed in claim 9 characterised in that the medicament is an adjuvant.

12. A flagellin protein or peptide fragment thereof for use in therapy characterised in that the protein or peptide fragment is truncated, mutated or has deletions therein which allow it to retain its ability to induce the immune response.

13. A flagellin protein or peptide fragment as claimed claim 12 characterised in that it retains the ability to bind to intestinal or epithelial cell flagellin receptors and retain immune signalling.

14. A flagellin protein or peptide fragment as claimed in claim 12 characterised in that the flagellin protein includes at least one of the conserved regions of residues 1-190 and 354-494 of S. typhimurium as shown underlined in FIG. 8 herein.

15. An adjuvant composition comprising a flagellin protein or peptide fragment thereof as claimed in claim 12 together with a pharmaceutically acceptable carrier, excipient or diluent in sterile pyrogen free form.

16. A vaccine composition comprising an adjuvant composition as claimed in claim 15 and a target antigen.
